(19) United States
(12) Patent Application Publication
(10) Pub. No.: US 2026/0311870 A1
(43) Pub. Date: Oct. 8, 2026

(54) SYSTEMS AND METHODS FOR DETERMINING A CUTANEOUS LESION SCORE OF A COMPANION ANIMAL

(71) Applicant: MARS, INCORPORATED, McLean, VA (US)

(72) Inventors: Xavier LANGON, Aimargues (FR); Mathieu MONTOYA, Aimargues (FR); Isabelle GOURDON, Aimargues (FR)

(21) Appl. No.: 19/164,425

(22) PCT Filed: Mar. 7, 2024
(86) PCT No.: PCT/US2024/018864

§ 371 Date: Sep. 11, 2025

(30) Foreign Application Priority Data
Mar. 14, 2023 (EP) 23161761.4

(51) Int. Cl. G16H 50/20; G16H 10/60; G16H 50/30; G16H 50/70

(52) U.S. Cl. CPC G16H 50/20; G16H 10/60; G16H 50/30; G16H 50/70

(57) ABSTRACT

The disclosure relates to a method for determining a cutaneous lesion score of a companion animal, using an assessment module trained based on previously acquired metadata relative to companion animals, selected in a data group having at least animal data and derm area data, the method including at least the steps of receiving metadata relative to said companion animal, including two or more animal data and two or more derm area data listed in the data group, encoding said metadata into a metadata vector, operating said trained assessment module on said metadata vector, based on an analysis of said metadata vector by said assessment module, generating a cutaneous lesion score indicative of the cutaneous state of said companion animal, and based on the cutaneous lesion score indicative of the at least one cutaneous state of said companion animal, assessing whether said companion animal has an atopic dermatitis condition.

### CROSS-REFERENCE TO RELATED APPLICATIONS

This application claims the benefit of priority of European Patent Application No. EP23161761.4, filed Mar. 14, 2023, the content of which is incorporated herein by reference in its entirety, and to which priority is claimed.

### FIELD OF THE DISCLOSURE

The disclosure relates to companion animal health, and in particular to the study of atopic dermatitis conditions. Various embodiments of this disclosure relate generally to machine-learning based techniques for monitoring companion animal health, and, more particularly, to systems and methods for determining a cutaneous lesion score of a companion animal suspected to have an atopic dermatitis condition.

### BACKGROUND

In most companion animals, a healthy skin and coat indicates that an animal is generally in good health. As the skin and coat condition of a companion animal provides such an important visual impact, in particular to their owners, it is an ongoing aim in the art to assess the cutaneous state of an animal.

A companion animal having a skin condition is the single most common reason for a companion animal to be taken to a veterinary practice. It is suggested that 15% of the workload of such veterinary practices accounts for animals suffering from skin conditions. One of the main skin conditions that contributes to this statistic is atopic dermatitis, which is a common, genetically predisposed, inflammatory and pruritic skin disease. The diagnosis of atopic dermatitis may be complicated by variables such as variations in its clinical presentations, genetic factors, extent of the lesions, stage of the disease, secondary infections, as well as resemblance to other non-atopic related skin diseases.

It can be difficult to reliably classify cutaneous lesions and efficiently guide the owner of the companion animal. Dermatology is a complex medical territory as skin can reveal all the external and internal diseases. A clinical examination is usually needed to diagnose a skin condition, which is time-consuming process that requires a deep understanding of dermatology. Furthermore, usually, only symptomatic treatments are prescribed for the skin condition, which results in not treating the underlying skin condition.

A smartphone application, named “Atopy index”, has been developed based on Canine Atopic Dermatitis Lesion Index (CADLI) and pruritus intensity. Such an application helps veterinarian practitioners to easily and quickly assess the Canine Atopic Dermatitis Extent and Severity Index (CADESI-4) before and after treatment. The owner of the companion animal identifies the lesions by coloring them on the application, which is based on 20 different areas of the body surface and 3 lesion types. The application allows obtaining the dermatological index in just a few minutes based on a validated severity scale, along with the severity index, and allows tracking the evolution of the total score and severity for each affected area at each visit. This application, however, is not designed for the diagnosis of atopic dermatitis, but only as an aid in its management for the owner.

It is described in Favrot et al. A prospective study on the clinical features of chronic canine atopic dermatitis and its diagnosis; Vet Dermatol; 2010; 21(1): 23-31: a method using several criteria, including derm area data, to assess the cutaneous state of a companion animal. The user has to use said criteria and further to recognize the pattern of the skin condition on the patient, which is variable depending on the animal breed.

Thus, there still remains a need for improving existing methods in the field of atopic dermatitis conditions, and for providing efficient, easy and reliable methods to assist an animal owner and/or a veterinarian practitioner in assessing the cutaneous state of a companion animal.

Reference to any particular activity is provided in this disclosure only for convenience and not intended to limit the disclosure. A person of ordinary skill in the art would recognize that the concepts underlying the disclosed devices and methods may be utilized in any suitable activity. The disclosure may be understood with reference to the following description and the appended drawings, wherein like elements are referred to with the same reference numerals.

The terminology used below may be interpreted in its broadest reasonable manner, even though it is being used in conjunction with a detailed description of certain specific examples of the present disclosure. Indeed, certain terms may even be emphasized below; however, any terminology intended to be interpreted in any restricted manner will be overtly and specifically defined as such in this detailed description section. Both the foregoing general description and the following detailed description are exemplary and explanatory only and are not restrictive of the features, as claimed.

### SUMMARY

According to certain aspects of the disclosure, methods and systems are disclosed for determining a cutaneous lesion score of a companion animal.

According to a first aspect, the disclosure relates to a method for determining a cutaneous lesion score of a companion animal suspected to have an atopic dermatitis condition, using an assessment module trained beforehand to learn features indicative of an atopic dermatitis condition in a companion animal, based at least on a plurality of previously acquired metadata relative to companion animals, selected in a data group (A) including at least animal data and derm area data, the method including at least the steps of:
a) receiving metadata relative to said companion animal, including two or more animal data and two or more derm area data listed in the data group (A),
b) encoding said metadata into a metadata vector,
c) operating said trained assessment module on said metadata vector, wherein the operating comprises inputting said metadata vector into a predictive model,
d) based on an analysis of said metadata vector by said predictive model associated with said assessment module, generating a cutaneous lesion score indicative of at least one cutaneous state of said companion animal, and
e) based on the cutaneous lesion score indicative of the at least one cutaneous state of said companion animal, assessing whether said companion animal has an atopic dermatitis condition.

By crossing the animal data and derm area data from the metadata, and by using said assessment module, trained beforehand, a score indicative of at least one cutaneous state of said companion animal can be obtained. The disclosure thus allows helping general veterinarian practitioners in their dermatologic diagnostic. The method can lead towards the dermatological diagnostic hypothesis that matches best with the clinical signs and give the chance to companion animals to have more reliable and faster diagnostics and treatments.

In an embodiment, the metadata relative to companion animals, selected in said data group (A), include at least animal data and derm area data, wherein:
animal data include two or more of at least (i) breed, (ii) species, (iii) gender, (iv) weight, (v) spayed or neutered status, (vi) age, (vii) age at onset, (viii) body condition, (ix) health status, (x) lifestyle, (xi) habitat, (xii) coat information, (xiii) activity level, (xiv) a biological value from a biological sample, (xv) age at onset and (xvi) dermatitis history; and
derm area data include two or more of at least (i) inguinal, (ii) axillar, (iii) ventral chest, (iv) perineal/genital, (v) ventral neck, (vi) pinnae, (vii) periorbitar, (viii) perioral, (ix) flexoral elbow, (x) fore feet, (xi) flexoral tarsal, (xii) hind feet and (xiii) nonspecific atopic area.

In a particular embodiment, the metadata relative to companion animals, selected in said data group (A), include at least animal data and derm area data, wherein:
animal data include at least (i) breed, (ii) species, (iii) lifestyle (iv) age at onset and (v) dermatitis history, and can further include other data such as, without limitation, gender, weight, spayed or neutered status, age, body condition, health status, habitat, coat information, activity level, and a biological value from a biological sample; and
derm area data include at least (i) inguinal, (ii) axillar and (iii) nonspecific atopic area, and can further include additional data such as, without limitation, ventral chest, perineal/genital, ventral neck, pinnae, periorbitar, perioral, flexoral elbow, fore feet, flexoral tarsal and hind feet,

In a particular embodiment, the metadata relative to companion animals, selected in said data group (A), include at least animal data and derm area data, wherein:
animal data include at least (i) breed, (ii) species, (iii) lifestyle (iv) age at onset and (v) dermatitis history; and
derm area data include at least (i) inguinal, (ii) axillar and (iii) nonspecific atopic area.

In a particular embodiment, the method further includes the steps of receiving a description of a pattern associated with a lesion of said companion animal and inputting the description into the predictive model. Generating the cutaneous lesion score is further based on an analysis of the description by the predictive model.

In a particular embodiment, the method further includes the steps of calculating second-or-more order cross-features based on cross feature interactions between said metadata and inputting the second-or-more order cross-features into the predictive model. Generating the cutaneous lesion score is further based on an analysis of the second-or-more order cross-features by the predictive model.

According to another aspect, the disclosure relates to a method for training an assessment module to learn features indicative of an atopic dermatitis condition in a companion animal, using at least a plurality of previously acquired metadata relative to companion animals, selected in a data group (A) including at least animal data including two or more of at least (i) breed, (ii) species, (iii) gender, (iv) weight, (v) spayed or neutered status, (vi) age, (vii) age at onset, (viii) body condition, (ix) health status, (x) lifestyle, (xi) habitat, (xii) coat information, (xiii) activity level, (xiv) a biological value from a biological sample, (xv) age at onset and (xvi) dermatitis history, and derm area data corresponding to affected or unaffected derm area including two or more of at least (i) inguinal, (ii) axillar, (iii) ventral chest, (iv) perineal/genital, (v) ventral neck, (vi) pinnae, (vii) periorbitar, (viii) perioral, (ix) flexoral elbow, (x) fore feet, (xi) flexoral tarsal, (xii) hind feet and (xiii) nonspecific atopic area, the method including:
extracting at least one feature from each previously acquired metadata,
associating at least said at least one feature to an animal cutaneous state, and
training the assessment module to learn said association.

According to another aspect, the present disclosure relates to a device for determining a cutaneous lesion score of a companion animal suspected to have an atopic dermatitis condition, the device including an assessment module trained beforehand to learn features indicative of an atopic dermatitis condition in a companion animal, based at least on a plurality of previously acquired metadata relative to companion animals, selected in a data group (A) including at least animal data including two or more of at least (i) breed, (ii) species, (iii) gender, (iv) weight, (v) spayed or neutered status, (vi) age, (vii) age at onset, (viii) body condition, (ix) health status, (x) lifestyle, (xi) habitat, (xii) coat information, (xiii) activity level, (xiv) a biological value from a biological sample, (xv) age at onset and (xvi) dermatitis history, and derm area data corresponding to affected or unaffected derm area including two or more of at least (i) inguinal, (ii) axillar, (iii) ventral chest, (iv) perineal/genital, (v) ventral neck, (vi) pinnae, (vii) periorbitar, (viii) perioral, (ix) flexoral elbow, (x) fore feet, (xi) flexoral tarsal, (xii) hind feet and (xiii) nonspecific atopic area, said trained assessment module being configured to operate on metadata relative to said companion animal, including two or more animal data and two or more derm area data listed in the data group (A), and to generate a cutaneous lesion score indicative of at least one cutaneous state of said companion animal.

The disclosure thus provides an efficient and reliable digital tool to help veterinarian practitioners in their diagnostic.

According to another aspect, the disclosure relates to a computer program product for determining a cutaneous lesion score of a companion animal suspected to have an atopic dermatitis condition, using an assessment module trained beforehand to learn features indicative of an atopic dermatitis condition in a companion animal, based at least on a plurality of previously acquired metadata relative to companion animals, selected in a data group (A) having at least animal data including two or more of at least (i) breed, (ii) species, (iii) gender, (iv) weight, (v) spayed or neutered status, (vi) age, (vii) age at onset, (viii) body condition, (ix) health status, (x) lifestyle, (xi) habitat, (xii) coat information, (xiii) activity level, (xiv) a biological value from a biological sample, (xv) age at onset and (xvi) dermatitis history, and derm area data corresponding to affected or unaffected derm area including two or more of at least (i) inguinal, (ii) axillar, (iii) ventral chest, (iv) perineal/genital, (v) ventral neck, (vi) pinnae, (vii) periorbitar, (viii) perioral, (ix) flexoral elbow, (x) fore feet, (xi) flexoral tarsal, (xii) hind feet and (xiii) nonspecific atopic area, the computer program product including a support and stored on this support instructions that can be read by a processor, these instructions being configured to:
a) receiving metadata relative to said companion animal, including two or more animal data and two or more derm area data listed in the data group (A),
b) encoding said metadata into a metadata vector,
c) operating said trained assessment module on said metadata vector, wherein the operating comprises inputting said metadata vector into a predictive model,
d) based on an analysis of said metadata vector by said predictive model associated with said assessment module, generating a cutaneous lesion score indicative of at least one cutaneous state of said companion animal, and
e) e) based on the cutaneous lesion score indicative of the at least one cutaneous state of said companion animal, assessing whether said companion animal has an atopic dermatitis condition.

According to another aspect, the disclosure relates to a computer-readable medium having stored thereon the computer program product as defined above.

According to an exemplary embodiment, the disclosure relates to a computer-implemented method for determining a cutaneous lesion score of a companion animal, including:
receiving metadata relative to said companion animal, including two or more animal data and two or more derm area data listed in the data group (A);
encoding said metadata into a metadata vector;
operating a trained assessment module on said metadata vector, wherein the operating comprises inputting said metadata vector into a predictive model;
based on an analysis of said metadata vector by said predictive model associated with said assessment module, generating a cutaneous lesion score indicative of at least one cutaneous state of said companion animal;
based on the cutaneous lesion score indicative of the at least one cutaneous state of said companion animal, assessing whether said companion animal has an atopic dermatitis condition; and
transmitting the cutaneous lesion score and the assessment to a device.

In an embodiment of the computer-implemented method according to the disclosure, the cutaneous lesion score assesses at least one other dermatitis condition, wherein the at least one other dermatitis condition can include at least one of: sarcoptic mange, demodicosis, bacterial overgrowth syndrome, Malassezia dermatitis, bacterial folliculitis, contact dermatitis, and/or mucocutaneous T cell lymphoma.

In an embodiment of the computer-implemented method according to the disclosure, the at least one cutaneous state can include at least one atopic dermatitis condition.

In an embodiment of the disclosure, the computer-implemented method can further include:
retrieving a previously stored cutaneous score;
comparing the previously stored cutaneous score to the cutaneous lesion score; and
based on the comparing, assessing a previous treatment curative effect.

In an embodiment of the disclosure, the computer-implemented can further include:
determining a recommendation based on the cutaneous lesion score; and
transmitting the recommendation to the device.

In an embodiment of the computer-implemented method according to the disclosure, the recommendation can be at least one health recommendation, at least one nutritional recommendation, and/or at least one medical recommendation.

In an embodiment of the computer-implemented method according to the disclosure, the at least one health recommendation can include at least one of: at least one food product, at least one pet service, at least one supplement, at least one ointment, at least one drug, and/or at least one pet product.

In an embodiment of the computer-implemented method according to the disclosure, the at least one nutritional recommendation can include at least one instruction to feed the companion animal at least one of: at least one supplement and/or at least one food.

In an embodiment of the computer-implemented method according to the disclosure, the at least one medical recommendation can include at least one of: at least one ointment instruction and/or at least one drug instruction.

In an embodiment of the computer-implemented method according to the disclosure, the trained assessment module can include at least one supervised classifier machine learning model.

In an embodiment of the computer-implemented method according to the disclosure, the training of the trained assessment module can further include:
updating a weight of the at least one supervised classifier machine learning model according to the associating between the animal cutaneous state and the at least one feature.

It is to be understood that both the foregoing summary and the following description are exemplary and explanatory only and are not restrictive of the disclosed embodiments, as claimed.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1 depicts a flowchart of an exemplary method of using a trained machine-learning model to determine a cutaneous lesion score for a companion animal suspected to have an atopic dermatitis condition according to one or more embodiments.

FIG. 2 shows an example of use of the method according to one or more embodiments, depicting an example of a user interface allowing for input data, such as companion animal metadata.

FIG. 3 depicts, according to several breeds, an example of some of the most commonly affected areas in a dog with atopic dermatitis condition, according to one or more embodiments.

FIG. 4 depicts an example of some of the most commonly affected areas in a dog with atopic dermatitis condition, according to one or more embodiments.

FIG. 5 depicts an example of some of the most commonly affected areas in a cat with an atopic dermatitis condition, according to one or more embodiments.

FIG. 6 depicts an example of the training of a Random Forest Classification algorithm used in the invention.

FIG. 7 depicts an example of use of an assessment module according to the invention.

### DETAILED DESCRIPTION

### Definitions

In the detailed description herein, references to “embodiment,” “an embodiment,” “one non-limiting embodiment,” “in various embodiments,” etc., indicate that the embodiment(s) described can include a particular feature, structure, or characteristic, but every embodiment might not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. After reading the description, it will be apparent to one skilled in the relevant art(s) how to implement the disclosure in alternative embodiments.

In general, terminology can be understood at least in part from usage in context. For example, terms, such as “and”, “or”, or “and/or,” as used herein can include a variety of meanings that man depend at least in part upon the context in which such terms are used. Typically, “or” if used to associate a list, such as A, B or C, is intended to mean A, B, and C, here used in the inclusive sense, as well as A, B or C, here used in the exclusive sense. In addition, the term “one or more” as used herein, depending at least in part upon context, can be used to describe any feature, structure, or characteristic in a singular sense or can be used to describe combinations of features, structures or characteristics in a plural sense.

Similarly, terms, such as “a,” “an,” or “the,” again, can be understood to convey a singular usage or to convey a plural usage, depending at least in part upon context. In addition, the term “based on” can be understood as not necessarily intended to convey an exclusive set of factors and can, instead, allow for existence of additional factors not necessarily expressly described, again, depending at least in part on context.

The terms “having,” “including,” “containing” and “comprising” or any other variation thereof, are interchangeable, and one of skill in the art will recognize that these terms are open ended terms. They are intended to cover a non-exclusive inclusion, such that a process, method, article, or apparatus that comprises a list of elements does not include only those elements but can include other elements not expressly listed or inherent to such process, method, article, or apparatus.

As used herein, the term “about” or “approximately” means within an acceptable error range for the particular value as determined by one of ordinary skill in the art, which will depend in part on how the value is measured or determined, i.e., the limitations of the measurement system.

As used herein, the term “companion animal” or “pet” refers to a non-human animal and may include, for example, without limitation, a companion mammal. For example, companion mammals may encompass canines, felines, dogs, cats, rabbits, hamsters, guinea pigs, rats and/or mice. Preferred companion animals herein, without limitation, are canine or feline, especially as dogs and cats, in particular dogs.

As used herein, the term “mammal” or “mammals” may include, for example, without limitation, a human or an animal. In particular, the term “animal” or “animals” may include, for example, without limitation, a ruminant, a poultry, a swine, a mammal, a horse, a mouse, a rat, a rabbit, a guinea pig, a hamster, a cow, a cat, or a dog, preferably a companion animal, i.e., a cat and/or a dog.

As used herein, the term “adult” may include, for example, without limitation, an animal that has passed puberty or has reached its biological maturation point, or both.

As used herein, the term “canine” may include animals, including companion animals selected from recognized dog breeds (some of which are further subdivided), which may include Afghan hound, airedale, akita, Alaskan malamute, basset hound, beagle, Belgian shepherd, bloodhound, border collie, border terrier, borzoi, boxer, bulldog, French bulldog, bull terrier, bullmastiff, cairn terrier, chihuahua, chow, cocker spaniel, collie, corgi, dachshund, dalmatian, doberman, shar pei, English setter, fox terrier, German shepherd, golden retriever, great Dane, greyhound, griffon bruxellois, Irish setter, Irish wolfhound, King Charles spaniel, Labrador retriever, lhasa apso, mastiff, newfoundland, old English sheepdog, papillion, Pekingese, pointer, pomeranian, poodle, pug, rottweiler, St. Bernard, saluki, samoyed, schnauzer, Scottish terrier, Shetland sheepdog, shih tzu, Siberian husky, Skye terrier, vizsla, Rhodesian, Staffordshire terrier, Jack terrier, springer spaniel, West Highland terrier, whip companion, Yorkshire terrier, bichon, etc.

As used herein, the term “feline” may include animals, including companion animals, selected from, without limitation, cheetah, puma, jaguar, leopard, lion, lynx, liger, tiger, panther, bobcat, ocelot, smilodon, caracal, serval and cats. As used herein, cats encompass wild cats and domestic cats, and most preferably domestic cats.

As used herein, a “subpopulation” may include, for example, without limitation, a set of one to several animals of one species, but less than an entire species. For example, “subpopulation” may be definable in terms of genotype and/or one or more attributes of physiological condition that, in a subpopulation of more than one member, are common to members of the subpopulation. In certain embodiments, the subpopulation may be defined at least in part by specific breed. Additionally, for example, in the case of animals of mixed breed, a subpopulation can be defined at least in part by breed heritage, which may be established through knowledge of the parental breeds, phenotypic characteristics, genotypic assessment, or by genetic markers such as SNPs. In certain embodiments, the subpopulation may be defined at least in part by physiological condition.

As used herein, the term “metadata” or “metadata file” may include any companion animal data, for example, without limitation, any one or combination of attributes of an animal including at least its breed, species, activity level, medical history, reproductive status, age, gender, weight, spayed or neutered status, a biological value from a biological sample, body condition, health status, lifestyle, habitat, coat information, or risk factor, and/or medical data, such as the age of disease onset, the existence of previous episodes of hotspots, urticarial or angioedema, the presence of cortico-response pruritus, the excess of hair loss, scaling or dryness, gastrointestinal signs, an indication on whether or not symptoms worsen after walking in grass, and/or medical history of chronic and/or recurrent dermatoses or otitis.

According to the disclosure, the term “animal data” may include two or more of at least (i) breed, (ii) species, (iii) gender, (iv) weight, (v) spayed or neutered status, (vi) age, (vii) age at onset, (viii) body condition, (ix) health status, (x) lifestyle, (xi) habitat, (xii) coat information, (xiii) activity level, (xiv) a biological value from a biological sample, (xv) age at onset and (xvi) dermatitis history. In a particular embodiment, animal data include at least (i) breed, (ii) species, (iii) lifestyle (iv) age at onset and (v) dermatitis history. In some embodiments, animal data include at least (i) breed, (ii) species, (iii) lifestyle (iv) age at onset and (v) dermatitis history, and can further include others data such as, without limitation, gender, weight, spayed or neutered status, age, body condition, health status, habitat, coat information, activity level, and a biological value from a biological sample; and

According to the specification, the term “derm area data” may include two or more of at least (i) inguinal, (ii) axillar, (iii) ventral chest, (iv) perineal/genital, (v) ventral neck, (vi) pinnae, (vii) periorbitar, (viii) perioral, (ix) flexoral elbow, (x) fore feet, (xi) flexoral tarsal, (xii) hind feet and nonspecific atopic area.
derm area data include at least (i) inguinal, (ii) axillar and (iii) nonspecific area, and can further include additional data such as, without limitation, ventral chest, perineal/genital, ventral neck, pinnae, periorbitar, perioral, flexoral elbow, fore feet, flexoral tarsal and hind feet,

According to the specification, the term “dermatological history” refers to the status of the dermatitis in the time, meaning if the dermatitis is a chronic dermatitis, a recurrent dermatitis or a permanent background.

According to the specification, the term “nonspecific atopic area” may include all derm area that are not (i) inguinal, (ii) axillar, (iii) ventral chest, (iv) perineal/genital, (v) ventral neck, (vi) pinnae, (vii) periorbitar, (viii) perioral, (ix) flexoral elbow, (x) fore feet, (xi) flexoral tarsal or (xii) hind feet.

Additionally, for example, the metadata may be composed of answers to questions pertaining to, but not limited to, the list of attributes above.

As used herein, the term “assessment module” may include, for example, without limitation, a module associated with a learning model and learning algorithms that analyze data, used in particular for classification and regression analysis.

As used herein, the term “expert” may include, for example, without limitation, a person able to identify cutaneous lesions on an animal body surface, to label features associated to such lesions, to discriminate such lesions and/or to associate them to an atopic dermatitis condition, other skin conditions, or other skin marks. Such an expert may be, for example, a veterinarian practitioner.

As used herein, the term “biological sample” or “biological material” may include, for example, without limitation, at least one of stool, urine, hair, blood, saliva, and tissue.

For example, the term “biological sample” or “biological material” may refer to a sample of tissue or fluid isolated from a subject, including but not limited to, for example, blood, plasma, serum, fecal matter, urine, bone marrow, bile, spinal fluid, lymph tissue and lymph fluid, samples of the skin, external secretions of the skin, respiratory, intestinal, and genitourinary tracts, tears, saliva, milk, blood cells, organs and/or biopsies. The term “biological sample” or “biological material” may also refer to samples of in vitro cell culture constituents including but not limited to conditioned media resulting from the growth of cells and tissues in culture medium, e.g., recombinant cells, and cell components. The term “biological sample” or “biological material” can also refer to, for example, without limitation, a polypeptide or a polynucleotide, or fragmented portions of organisms or cells obtained from sampling the environment, such as airborne pathogens.

As used herein, the term “food product” or “food composition” or “diet” or “foodstuff” may refer to, for example, without limitation foodstuff, diet, food supplement, liquid and/or a material that may contain proteins, carbohydrates and/or crude fats. For example, the term may also refer to supplementary substances or additives, for example, minerals, vitamins, and condiments (See Merriam-Webster's Collegiate Dictionary, 10th Edition, 1993, the entirety of which is hereby incorporated herein by reference). Such food compositions or products may be nutritionally complete or not.

As used herein, a “companion food” or “animal food” may include, for example, without limitation, a product produced by a companion food manufacturer, whether processed, partially processed or unprocessed, and/or intended to be ingested by companion animals after placing on the market; according to European Union (EU) Regulation n°767/2009.

As used herein, a “training data set” or “training data” can include one or more data to train a machine learning model. A training data set can be collected via one or more client devices (e.g., crowd-sourced) or collected from other sources (e.g., a database). In certain non-limiting embodiments, the training data set for a wellness assessment of a pet can include data from both a treatment group and a control group.

Certain non-limiting embodiments are described below with reference to block diagrams and operational illustrations of methods, processes, devices, and apparatus. It is understood that each block of the block diagrams or operational illustrations, and combinations of blocks in the block diagrams or operational illustrations, can be implemented by means of analog or digital hardware and computer program instructions. These computer program instructions can be provided to a processor of a general-purpose computer to alter its function as detailed herein, a special purpose computer, ASIC, or other programmable data processing apparatus, such that the instructions, which execute via the processor of the computer or other programmable data processing apparatus, implement the functions/acts specified in the block diagrams or operational block or blocks. In some alternate implementations, the functions/acts noted in the blocks can occur out of the order noted in the operational illustrations. For example, two blocks shown in succession can in fact be executed substantially concurrently or the blocks can sometimes be executed in the reverse order, depending upon the functionality/acts involved.

In certain non-limiting embodiments, the term “server” should be understood to refer to a service point which provides processing, database, and communication facilities. By way of example, and not limitation, the term “server” can refer to a single, physical processor with associated communications and data storage and database facilities, or it can refer to a networked or clustered complex of processors, such as an elastic computer cluster, and associated network and storage devices, as well as operating software and one or more database systems and application software that support the services provided by the server. The server, for example, can be a cloud-based server, a cloud-computing platform, or a virtual machine. Servers can vary widely in configuration or capabilities, but generally a server can include one or more central processing units and memory. A server can also include one or more mass storage devices, one or more power supplies, one or more wired or wireless network interfaces, one or more input/output interfaces, or one or more operating systems, such as Windows Server, Mac OS X, Unix, Linux, FreeBSD, or the like.

For some non-limiting embodiments, a “network” should be understood to refer to a network that may couple devices so that communications can be exchanged, such as between a server and a client device or other types of devices, including between wireless devices coupled via a wireless network, for example. A network can also include mass storage, such as network attached storage (NAS), a storage area network (SAN), or other forms of computer or machine-readable media, for example. A network can include the Internet, one or more local area networks (LANs), one or more wide area networks (WANs), wire-line type connections, wireless type connections, cellular or any combination thereof. Likewise, sub-networks, which can employ differing architectures or can be compliant or compatible with differing protocols, can interoperate within a larger network. Various types of devices can, for example, be made available to provide an interoperable capability for differing architectures or protocols. As one illustrative example, a router can provide a link between otherwise separate and independent LANs.

A communication link or channel can include, for example, analog telephone lines, such as a twisted wire pair, a coaxial cable, full or fractional digital lines including T1, T2, T3, or T4 type lines, Integrated Services Digital Networks (ISDNs), Digital Subscriber Lines (DSLs), wireless links including satellite links, or other communication links or channels, such as can be known to those skilled in the art. Furthermore, a computing device or other related electronic devices can be remotely coupled to a network, such as via a wired or wireless line or link, for example.

In certain non-limiting embodiments, a “wireless network” should be understood to couple client devices with a network. A wireless network can employ standalone ad-hoc networks, mesh networks, wireless land area network (WLAN), cellular networks, or the like. A wireless network may be configured to include a system of terminals, gateways, routers, or the like coupled by wireless radio links, or the like, which can move freely, randomly, or organize themselves arbitrarily, such that network topology can change, at times even rapidly. A wireless network can further employ a plurality of network access technologies, including Wi-Fi, Long Term Evolution (LTE), WLAN, Wireless Router (WR) mesh, or 2nd, 3rd, 4th, 5th generation (2G, 3G, 4G, or 5G) cellular technology, or the like. Network access technologies can allow wide area coverage for devices, such as client devices with varying degrees of mobility, for example. For example, a network may be configured to provide radio frequency (RF) or wireless type communication via one or more network access technologies, such as Global System for Mobile communication (GSM), Universal Mobile Telecommunications System (UMTS), General Packet Radio Services (GPRS), Enhanced Data GSM Environment (EDGE), 3GPP LTE, LTE Advanced, Wideband Code Division Multiple Access (WCDMA), Bluetooth, 802.11b/g/n, or the like. A wireless network can include virtually any type of wireless communication mechanism by which signals can be communicated between devices, such as a client device or a computing device, between or within a network, or the like. A computing device can send or receive signals, such as via a wired or wireless network, or can process or store signals, such as in memory as physical memory states. For example, a computing device can operate as a server and can include, as examples, dedicated rack-mounted servers, desktop computers, laptop computers, set top boxes, integrated devices combining various features, such as two or more features of the foregoing devices, or the like. Servers can vary widely in configuration or capabilities, but generally a server can include one or more central processing units and memory. A server can also include one or more mass storage devices, one or more power supplies, one or more wired or wireless network interfaces, one or more input/output interfaces, or one or more operating systems.

As used herein, a “machine-learning model” generally encompasses instructions, data, and/or a model configured to receive input, and apply one or more of a weight, bias, classification, or analysis on the input to generate an output. The output may include, for example, a classification of the input, an analysis based on the input, a design, process, prediction, or recommendation associated with the input, or any other suitable type of output. A machine-learning model is generally trained using training data, e.g., experiential data and/or samples of input data, which are fed into the model in order to establish, tune, or modify one or more aspects of the model, e.g., the weights, biases, criteria for forming classifications or clusters, or the like. Aspects of a machine-learning model may operate on an input linearly, in parallel, via a network (e.g., a supervised classifier machine learning model such as, without limitation), or via any suitable configuration.

The execution of the machine-learning model may include deployment of one or more classifier machine learning techniques or models, such as linear models (including for example logistical regression), ensemble methods (including for example random forest classifier), gradient boosted machine (GBM), discriminant Analysis (including for example Patial Least Square Discriminant Analysis), Support Vector Classifier, Nearest Neighbors (including for example K-Nearest Neighbors Classifier), Gaussian Processes Classifier, Naive Bayes (including for example Gaussian Naive Bayes Classifier), Decision Trees Classifier, Neural network models (including for example Multi-layer Perceptron Classifier), deep learning, and/or a deep neural network. Supervised training may be employed. For example, supervised learning may include providing training data and labels corresponding to the training data, e.g., as ground truth. Any suitable type of training may be used, e.g., stochastic, gradient boosted, random seeded, recursive, epoch or batch-based, etc. In an exemplary use case, a machine-learning model may determine a cutaneous lesion score of a companion animal. The computer system may be configured to first receive companion animal data corresponding to the companion animal. For example, the companion animal data may include at least one metadata file. A trained assessment module may then analyze, or operate on, the companion animal data, where the trained assessment module generates a cutaneous lesion score based on the analyzing. The cutaneous lesion score may, for example, indicate at least one cutaneous state of the companion animal, where one of the at least one cutaneous states may include at least one atopic dermatitis condition. The computer system may then be configured to transmit the cutaneous lesion score to a device, which may belong to a veterinarian and/or the guardian of the companion animal. In another exemplary use case, a machine-learning model, such as a trained assessment module, may be trained to generate a cutaneous lesion score. The training may include utilizing training data. For example, the trained assessment module may extract at least one feature from each of the plurality of previously acquired metadata. The trained assessment module may then associate the at least one feature with a companion animal cutaneous state. This process may continue until an association may be made for each of the previously acquired metadata. The trained assessment module may then be further trained based on such associations.

While the examples above involve cutaneous lesions and atopic dermatitis conditions, it should be understood that techniques according to this disclosure may be adapted to any suitable type of companion animal health analysis. It should also be understood that the examples above are illustrative only. The techniques and technologies of this disclosure may be adapted to any suitable activity.

### Method for Determining a Cutaneous Lesion Score of a Companion Animal

According to an aspect, the disclosure relates to a method for determining a cutaneous lesion score of a companion animal suspected to have an atopic dermatitis condition, using an assessment module trained beforehand to learn features indicative of an atopic dermatitis condition in a companion animal, based at least on a plurality of previously acquired metadata relative to companion animals, selected in a data group (A) having at least animal data and derm area data, the method including at least the steps of:
a) receiving metadata relative to said companion animal, including two or more animal data and two or more derm area data listed in the data group (A),
b) operating said trained assessment module on said metadata received at step a), and
c) based on said assessment module, generating a cutaneous lesion score indicative of at least one cutaneous state of said companion animal.

In one embodiment, said companion animal is a canine or a feline.

In one embodiment, said companion animal is a dog or a cat.

In one embodiment, said companion animal is a dog.

### Metadata Relative to Companion Animals

In one embodiment, said metadata can include the breed of the animal, an indication about its lifestyle, especially if its indoor life is predominant or not, the age of atopic dermatitis condition onset, an indication about the fact that said condition is chronic, recurrent or a permanent background, and an indication about at least one affected derm area, especially the inguinal area, the axillar area or another area.

Said data group (A) can further include reproductive status, risk factor, and/or an indication about at least one affected area of the companion animal body surface, the existence of previous episodes of hotspots, urticaria or angioedema, the presence of cortico-response pruritus, the excess of hair loss, scaling or dryness, gastrointestinal signs, an indication on whether or not symptoms worsen after walking in grass, or medical history of chronic and/or recurrent dermatoses or otitis.

Said derm area data can be preferably chosen among the head, especially the ear, pinnae, periorbitar, perioral and/or periocular areas, the legs, especially the fore feet, hind feet and/or the interdigital areas, the flexoral elbow, the flexor surface of the tarsal joint and/or the extensor surface of the carpal joint, and the trunc, especially the groin, armpit, ventral chest or neck, inguinal, axillar and/or perineal/genital areas.

The metadata thus advantageously can come from disease history and animal characteristics. Their use in the method according to the disclosure can allow increasing the specificity and sensitivity of the method. The metadata can be composed of answers to questions asked to the owner and/or to the veterinarian practitioner.

The method according to the disclosure can include the step of providing a pathological profile of said companion animal based on said plurality of metadata. In some embodiments, the pathologic profile may comprise a readily retrievable, centrally located information record that contains said plurality of metadata of said companion animal.

### Assessment Module

In a particular embodiment, said assessment module can use a predictive model.

In a particular embodiment, said predictive model can include a supervised classifier machine learning model, and a metadata encoding module.

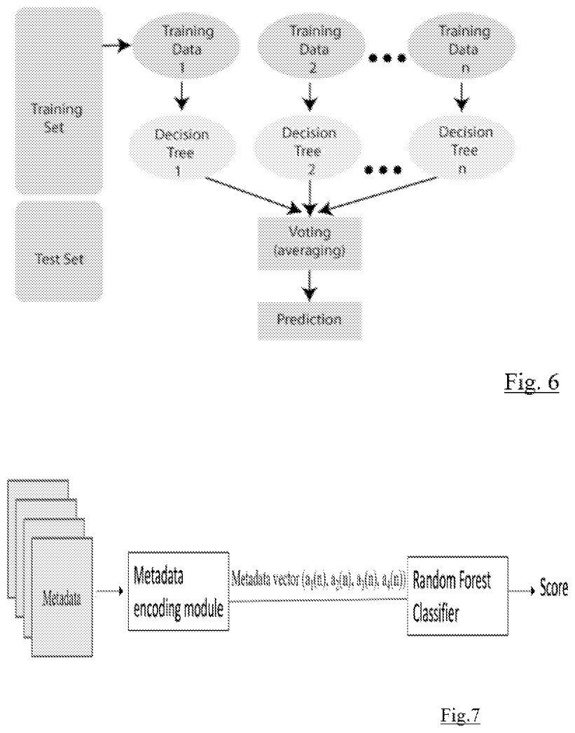

In a preferred embodiment, said model is a Random Forest Classification algorithm which is trained as explained below and as shown in FIG. 6. In such an embodiment, the full dataset is split in Training Set and Test Set, with:
Step 1: Select random samples from a given data or training set,
Step 2: Construction of a decision tree for every training data,
Step 3: Voting take place by averaging the decision tree or by majority voting decision tree, and
Step 4: Validation of the model performance with Test Set.

Other known classification algorithms may be used.

In other embodiments, said predictive model can include one or several neural networks.

Although particular embodiments use a training set and a test set, the predictive model could also be trained using a cross-validation methodology, that could be configured from 5 folds, up to 600 folds (one-leave one out cross validation).

Said metadata encoding module can be advantageously configured to convert said metadata, especially the answers to questions as defined above, into a categorical vector. The list of questions having a set of predefined answers, said metadata encoding module advantageously uses a binarization function to attribute a 1 or a 0 to each possible answer leading to a metadata vector.

In a particular embodiment, said assessment module can further include an assessment supervised classifier machine learning model

Said assessment supervised classifier machine learning model advantageously can use said metadata vector to generate the score indicative of the cutaneous state of said companion animal.

Said vector can be each associated to a companion animal and to the known cutaneous state of said animal.

The supervised classifier machine learning model can be refined for the training in order to minimize the errors and to generate the score on testing data or new incoming data. As an example and not by way of limitation, other features not selected in the model could be added to refine the model.

The pattern of the skin condition on the animal can be a variable depending on the animal breed. In a particular embodiment, said predictive model can use the description of the pattern of the lesion and the pre-disposed breed. The description of the pattern of the lesion and the pre-disposed breed may be two independent variables. If there is an effect of one influencing the other, and if such an effect has been captured in the dataset, the use of a random forest which can model non-linearity, would help to model this effect. A further improvement could be to add cross features interaction at an order 3 or 4, to generate new variables and use these new variables as additional input to the predictive model. In some embodiments, the computer-implemented method can further include providing second or more order cross-features calculated from the selected features. In some embodiments of the computer-implemented method, the cross-features can comprise third order cross-features.

### Cutaneous Lesion Score

Said score can be a probability, or a percentage, showing the level of confidence for the lesion(s) to be indicative of an atopic dermatitis condition, for example low, medium or high.

In an embodiment, said score can be in the form of a numerical value, for example a value of 0 (no dermatitis condition) or 1 (dermatitis condition).

In another embodiment, said score can be in the form of a numerical value, for example a value between 0 and 10. In another embodiment, said score can be in the form of a letter, especially showing that the cutaneous state of said companion animal is thought to belong to a type of cutaneous states, for example group A, group B, group C, and so on.

In yet another embodiment, said score can be a class indicative of the cutaneous state of said companion animal, or a class indicative of a dermatitis condition in said companion animal, atopic or otherwise, or a combination thereof, for example a class and a probability that the animal belongs to said class.

The method according to the disclosure can further include the step of providing to a user interface said score relative to said companion animal and generated at step c).

Said score(s) can be transmitted to a user by any suitable mean, for example by being displayed on a screen of an electronic device, printed, or by vocal synthesis.

Said score(s) can be used as entry value in another program, and/or maybe combined to other information, for example clinical and/or biological data.

The method according to the disclosure can suggest some complementary analysis to further refine the score. As an example and not by way of limitation, the score could be improved by adding new questions/inputs. A suggestion of the best diet associated with each score may also be provided.

Said score relative to said companion animal and generated at step c) can further assess the severity evolution of said atopic dermatitis condition. To assess the severity evolution of said atopic dermatitis condition, particular embodiments may add the size (e.g., in millimeters) on each lesion, which could be normalized by the size of the pet, so that a percentage of coverage of the lesion over the body of the pet can be assessed.

In an embodiment, if said score relative to said companion animal and generated at step c) assessing that said at least one cutaneous lesion is not indicative of an atopic dermatitis condition in said companion animal, said score assesses other dermatitis conditions such as sarcoptic mange, demodicosis, bacterial overgrowth syndrome, Malassezia dermatitis, bacterial folliculitis, contact dermatitis, or mucocutaneous T cell lymphoma. In such an embodiment, features and/or clinical criterions different than the ones used for atopic dermatitis condition can be used, in order to best describe such other dermatitis conditions.

In an embodiment, a previous score relative to the same companion animal being stored, the method can further include the step of comparing said previous score to the score generated at step c) in order to assess the curative effect of an adopted treatment method. According to another embodiment, the disclosure relates to a method for assessing the nature of cutaneous lesion(s) of a companion animal suspected to have an atopic dermatitis condition, using at least an assessment module trained beforehand to learn features indicative of an atopic dermatitis condition in a companion animal, based at least on a plurality of previously acquired metadata relative to companion animals, selected in a data group (A) including at least animal data and derm area data, the method including at least the steps of:
a) receiving metadata relative to said companion animal, including two or more animal data and two or more derm area data listed in the data group (A),
b) operating said trained assessment module on said metadata, and
c) based on said assessment module, generating a score assessing at least whether or not said metadata are indicative of an atopic dermatitis condition in said companion animal.

### Method for Training an Assessment Module to Learn Features Indicative of an Atopic Dermatitis Condition in a Companion Animal

According to another aspect, the disclosure relates to a method for training an assessment module to learn features indicative of an atopic dermatitis condition in a companion animal, using at least a plurality of previously acquired metadata relative to companion animals, selected in a data group (A) including at least animal data and derm area data, the method including:
extracting at least one feature from each previously acquired metadata,
associating at least said at least one feature to an animal cutaneous state, and
training the assessment module to learn said association.

In a particular embodiment, for the learning, not all data are of companion animals with cutaneous lesions, but some data can be of animals which are not affected with cutaneous lesions, and some can be of animals with lesions due to conditions other than atopic dermatosis. The global learning dataset preferably can contain atopic animals, animals affected with other dermatosis and healthy animals.

In one embodiment, said assessment module can include at least one supervised classifier machine learning model, the method including the step of updating weights of the supervised classifier machine learning model according to said association between said at least one feature and said animal cutaneous state.

The assessment module can be configured to assign weights to each of the inputs during the learning phase, the weights optimization being especially carried out by a solver of the “stochastic gradient descent” type.

In one embodiment, said association between said at least one feature and said animal cutaneous state can be used to modify atopic dermatitis features of reference for a future iteration of the method, in order to improve the accuracy and reliability of the method according to the disclosure.

The features described above for the method for determining a cutaneous lesion score can apply to the method for training an assessment module, and vice and versa.

### Devices & Computer-Implemented Methods & Mediums

The provided methods can be computer-implemented methods.

Hence, in one embodiment, the provided methods for determining a cutaneous lesion score of a companion animal suspected to have an atopic dermatitis condition and/or for training an assessment module to learn features indicative of an atopic dermatitis condition in a companion animal can be achieved either offline, i.e., not controlled by a device such as a computer-aided system; or alternatively online, i.e. controlled by a computer-aided system, such as one including a device suitable for determining a cutaneous lesion score of a companion animal suspected to have an atopic dermatitis condition, and having means adapted to execute the steps of the said method; or alternatively both offline and online.

Hence, according to one embodiment, the disclosure relates to a device for determining a cutaneous lesion score of a companion animal suspected to have an atopic dermatitis condition, the device including an assessment module trained beforehand to learn features indicative of an atopic dermatitis condition in a companion animal, based at least on a plurality of previously acquired metadata relative to companion animals, selected in a data group (A) including at least animal data and derm area data, said trained assessment module being configured to operate on metadata relative to said companion animal, including two or more animal data and two or more derm area data listed in the data group (A), and to generate a cutaneous lesion score indicative of at least one cutaneous state of said companion animal.

Said device can include an acquisition module for acquiring said metadata.

According to another aspect, the disclosure relates to a device for training an assessment module according to the method for training an assessment module to learn features indicative of an atopic dermatitis condition in a companion animal, as described above.

According to another embodiment, the disclosure relates to a computer program product for determining a cutaneous lesion score of a companion animal suspected to have an atopic dermatitis condition, using an assessment module trained beforehand to learn features indicative of an atopic dermatitis condition in a companion animal, based at least on a plurality of previously acquired metadata relative to companion animals, selected in a data group (A) including at least animal data and derm area data, the computer program product including a support and stored on this support instructions that can be read by a processor, these instructions being configured to:
a) receiving metadata relative to said companion animal, including two or more animal data and two or more derm area data listed in the data group (A),
b) operating said trained assessment module on said metadata received at step a), and
c) based on said assessment module, generating a cutaneous lesion score indicative of at least one cutaneous state of said companion animal.

According to another embodiment, the disclosure relates to a computer program product for training an assessment module according to the method for training an assessment module to learn features indicative of an atopic dermatitis condition in a companion animal, as described above.

According to another embodiment, the disclosure relates to a computer-readable medium having stored thereon one or both of the computer program products described above. Such a computer-readable medium can include, or can consist of, a physical embodiment of a collection of data including one to a plurality of data sets that can be configured in one to a plurality of databases. It can thus include, or can consist of, medium wherein or whereon such data can be stored. Such a computer-readable medium can also include or consist of more than one medium; however in such a case the media can be functionally linked.

A computer-aided system of the disclosure typically can include one or more user interface(s) enabling entry of an input data. Such input data can, for instance, include metadata relative to said companion animal.

Embodiments of the disclosure and all of the functional operations described in this specification can be implemented in digital electronic circuitry, or in computer software, firmware, or hardware, including the structures disclosed in this specification and their structural equivalents, or in combinations of one or more of them. Embodiments of the disclosure can be implemented as one or more computer program products, i.e., one or more modules of computer program instructions encoded on a computer-readable medium for execution by, or to control the operation of, data processing apparatus.

The computer-readable medium can be a machine-readable storage device, a machine-readable storage substrate, a memory device, a composition of matter effecting a machine-readable propagated signal, or a combination of one or more of them. The term “data processing apparatus” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them. A propagated signal is an artificially generated signal, e.g., a machine-generated electrical, optical, or electromagnetic signal, that is generated to encode information for transmission to suitable receiver apparatus.

A computer program (also known as a program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network

A computer can be embedded in another device, e.g., a mobile telephone, a personal digital assistant (PDA), a mobile audio player, a Global Positioning System (GPS) receiver, to name just a few. Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks.

To provide for interaction with a user, embodiments of the disclosure can be implemented on a computer or a smartphone having a display device for displaying information to the user and a keyboard and a pointing or tactile device by which the user can provide input to the computer.

Embodiments of the disclosure can be implemented in a computing system that can include a back-end component, e.g., as a data server, or that can include a middleware component, e.g., an application server, or that can include a front-end component, e.g., a client computer having a graphical user interface or a Web browser through which a user can interact with an implementation of the disclosure, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), e.g., the Internet.

According to a seventh aspect, the disclosure relates to a computer-implemented method for determining a cutaneous lesion score of a companion animal suspected to have an atopic dermatitis condition, the method including: receiving at least one metadata relative to said companion animal, determining based on these data a cutaneous lesion score of said companion animal and determining a recommendation to a pet owner based on the score. In a particular embodiment, said recommendation is a nutritional recommendation. In another particular embodiment, this method can further include a step of transmitting the recommendation to a mobile device of the pet owner.

Based on the score, a recommendation can be determined and transmitted to one or more of a pet owner, a veterinarian, a researcher and/or any combination thereof. The recommendation, for example, can include one or more health recommendations for preventing the companion animal from developing one or more of a disease, a condition, an illness and/or any combination thereof. The recommendation, for example, can include one or more of: a food product, a pet service, a supplement, an ointment, a drug to improve the wellness or health of the pet, a pet product, and/or any combination thereof. In other words, the recommendation can be a nutritional recommendation. In some embodiments, a nutritional recommendation can include an instruction to feed a companion animal one or more of: a chewable, a supplement, a food and/or any combination thereof. In some embodiments, the recommendation can be a medical recommendation. For example, a medical recommendation can include an instruction to apply an ointment to a companion animal, to administer one or more drugs to a companion animal and/or to provide one or more drugs for or to a companion animal. The term “pet product” can include, for example, without limitation, any type of product, service, or equipment that is designed, manufactured, and/or intended for use by a companion animal. For example, the pet product can be a toy, a chewable, a food, an item of clothing, a collar, a medication, a health tracking device, a location tracking device, and/or any combination thereof. In another example a pet product can include a genetic or DNA testing service for pets. The term “pet owner” can include any person, organization, and/or collection of persons that owns and/or is responsible for any aspect of the care of a companion animal.

### EXAMPLES

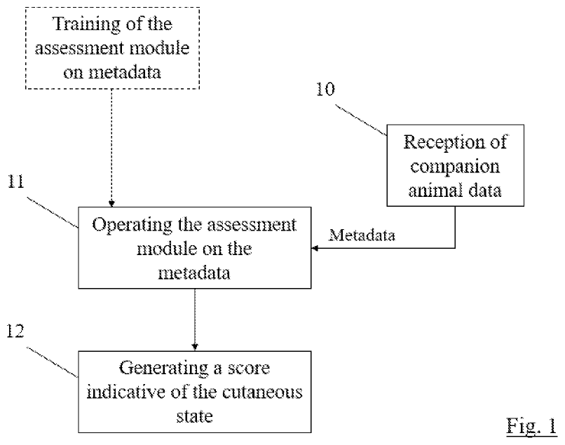

Various steps in an example of a method, according to the disclosure, for determining a cutaneous lesion score of a companion animal suspected to have an atopic dermatitis condition, are depicted in FIG. 1 and will be described in detail in what follows.

The method uses an assessment module trained beforehand to learn features indicative of an atopic dermatitis condition in a companion animal, based at least on a plurality of previously acquired metadata relative to companion animals. Said metadata are selected in a data group (A) having at least animal data and derm area data, wherein, in this example:
animal data include at least (i) breed, (ii) species, (iii) lifestyle (iv) age at onset and (v) dermatitis history, and
derm area data include at least (i) inguinal, (ii) axillar and (iii) nonspecific atopic area.

At a step 10 is received companion animal data corresponding to a companion animal, wherein the companion animal data include a plurality of metadata relative to said companion animal, including two or more animal data and two or more derm area data listed in said data group (A), and as described above.

During a step 11, said trained assessment module is operated on said at least one metadata of said companion animal. During a step 12, based on said assessment module, a score indicative of the cutaneous state of said companion animal is generated.

Some facultative steps can lead to a more reliable assessment, as the use of more data, more tests or questions.

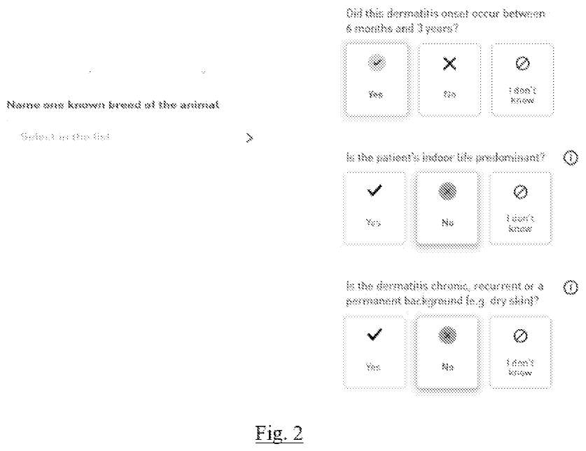

FIG. 2 shows an example of use of the method according to the disclosure through a smartphone application.

In this example, the user of the method according to the disclosure is asked to identify his dog's breed in a defined list of predisposed breeds. FIG. 2 shows a user interface enabling the animal owner to entry some input data, namely metadata relative to said companion animal such as its known breed, how long ago the alleged dermatitis onset occurred, indoor or outdoor life, chronic or recurrent dermatitis or behaving as a permanent background. Four animal data are thus used in this example.

The pet owner is also asked to choose the area(s) of its body surface showing the most characteristic lesion(s), for instance head, armpit, groin or foot. As explained above, all of these input data allow generating a score indicative of the cutaneous state of the animal, which may be shown on a subsequent interface. In this example, the user is asked to select the areas in a predefined list of 12 areas in order to form the derm area data.

The performances obtained in this example, with four animal data and fifteen derm area data, are summarized in the table below.

| Sensitivity | Specificity |
|---|---|
| 0.95 | 0.89 |
| PPV | NPV |
| 0.95 | 0.88 |

In a preferred embodiment, the assessment module uses a predictive model which include a supervised classifier machine learning model, and a metadata encoding module, as illustrated in FIG. 7.

For the training of the supervised classifier machine learning model of the assessment module, at least 100 companion animals with cutaneous lesions, some of healthy animals and some animals with features other than atopic dermatosis are used. The selection can include different ages, behaviors, all different colors of hair, cross-modification with aging, all different hair density and composition (primary/intermediate/secondary hairs). The distribution of animals has to be as large as possible to have the most important database.

In the illustrated example, the metadata encoding module is configured to convert said metadata, that is to say answers aᵢ(n) to questions qᵢ, into a categorical vector a₁(1) . . . aᵢ(n), with i the number of the question and n the number of predefined answers.

In the illustrated example, the assessment supervised classifier machine learning model uses said metadata vector of metadata encoding module to generate the score indicative of the cutaneous state of said companion animal, based on the vectors learned beforehand and associated with known animal cutaneous states.

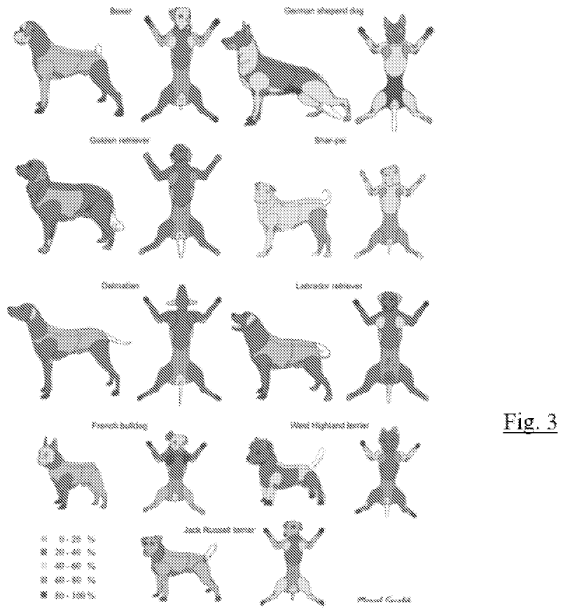

FIG. 3 shows some commonly areas in a dog affected with an atopic dermatitis condition, according to several common breeds. It can be noted that the breed has an impact on the areas affected by the condition.

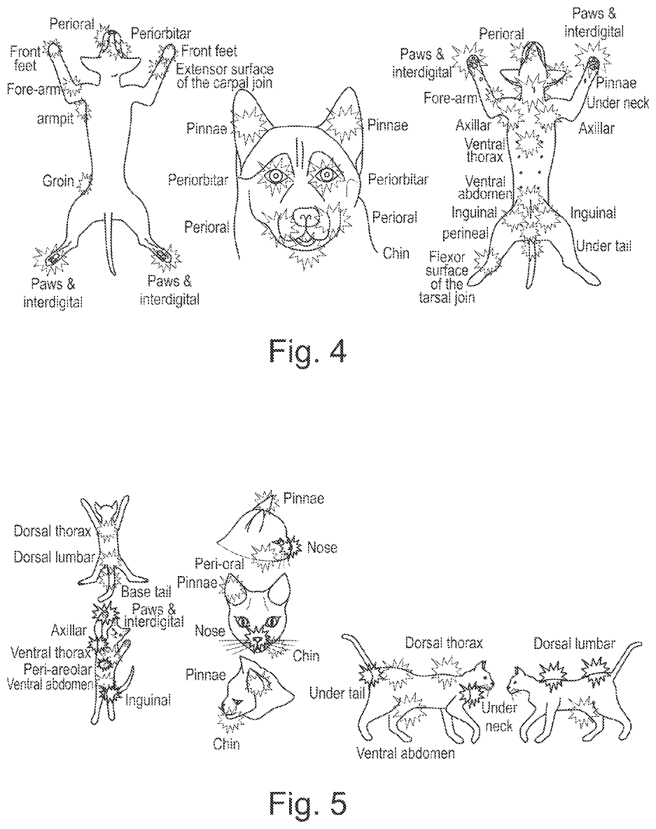

FIG. 4 shows some of the most commonly areas in a dog affected with an atopic dermatitis condition.

FIG. 5 shows some of the most commonly areas in a cat affected with an atopic dermatitis condition.

The most commonly affected areas are thus generally the head, especially the ear, perioral and/or periocular areas, the legs, especially the front feet and/or the interdigital areas, the flexor surface of the tarsal joint and/or the extensor surface of the carpal joint, and the trunc, especially the groin, armpit, ventral and/or perineal areas.

It should be appreciated that in the above description of exemplary embodiments of the disclosure, various features of the disclosure are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the disclosure requires more features than are expressly recited in each claim. Rather, as the following claims reflect, inventive aspects lie in less than all features of a single foregoing disclosed embodiment. Thus, the claims following the detailed description are hereby expressly incorporated into this detailed description, with each claim standing on its own as a separate embodiment of this disclosure.

Furthermore, while some embodiments described herein include some but not other features included in other embodiments, combinations of features of different embodiments are meant to be within the scope of the disclosure, and form different embodiments, as would be understood by those skilled in the art. For example, in the following claims, any of the claimed embodiments can be used in any combination.

Thus, while certain embodiments have been described, those skilled in the art will recognize that other and further modifications may be made thereto without departing from the spirit of the disclosure, and it is intended to claim all such changes and modifications as falling within the scope of the disclosure. For example, functionality may be added or deleted from the block diagrams and operations may be interchanged among functional blocks. Steps may be added or deleted to methods described within the scope of the present disclosure

The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description. While various implementations of the disclosure have been described, it will be apparent to those of ordinary skill in the art that many more implementations are possible within the scope of the disclosure. Accordingly, the disclosure is not to be restricted except in light of the attached claims and their equivalents

1. A method for determining a cutaneous lesion score of a companion animal suspected to have an atopic dermatitis condition, using an assessment module trained beforehand to learn features indicative of an atopic dermatitis condition in a companion animal, based at least on a plurality of previously acquired metadata relative to companion animals, selected in a data group (A) comprising at least animal data including two or more of at least (i) breed, (ii) species, (iii) gender, (iv) weight, (v) spayed or neutered status, (vi) age, (vii) age at onset, (viii) body condition, (ix) health status, (x) lifestyle, (xi) habitat, (xii) coat information, (xiii) activity level, (xiv) a biological value from a biological sample, (xv) age at onset and (xvi) dermatitis history, and derm area data corresponding to affected or unaffected derm area including two or more of at least (i) inguinal, (ii) axillar, (iii) ventral chest, (iv) perineal/genital, (v) ventral neck, (vi) pinnae, (vii) periorbitar, (viii) perioral, (ix) flexoral elbow, (x) fore feet, (xi) flexoral tarsal, (xii) hind feet and (xiii) nonspecific atopic area, the method comprising at least the steps of:
a) receiving metadata relative to said companion animal, including two or more animal data and two or more derm area data listed in the data group (A),
b) encoding said metadata into a metadata vector,
c) operating said trained assessment module on said metadata vector, wherein the operating comprises inputting said metadata vector into a predictive model,
d) based on an analysis of said metadata vector by said predictive model associated with said assessment module, generating a cutaneous lesion score indicative of at least one cutaneous state of said companion animal, and
e) based on the cutaneous lesion score indicative of the at least one cutaneous state of said companion animal, assessing whether said companion animal has an atopic dermatitis condition.

2. The method of claim 1, further comprising the step of:
receiving a description of a pattern associated with a lesion of said companion animal; and
inputting the description into the predictive model;
wherein generating the cutaneous lesion score is further based on an analysis of the description by the predictive model.

3. The method of claim 1, further comprising the steps of:
calculating second-or-more order cross-features based on cross feature interactions between said metadata; and
inputting the second-or-more order cross-features into the predictive model;
wherein generating the cutaneous lesion score is further based on an analysis of the second-or-more order cross-features by the predictive model.

4. The method of claim 1, wherein said data group (A) further includes reproductive status, risk factor, and/or an indication about at least one affected area of the companion animal body surface, the existence of previous episodes of hotspots, urticaria or angioedema, the presence of cortico-response pruritus, the excess of hair loss, scaling or dryness, gastrointestinal signs, an indication on whether or not symptoms worsen after walking in grass, or medical history of chronic and/or recurrent dermatoses or otitis.

5. The method of claim 1, further comprising the step of providing a pathological profile of said companion animal based on said plurality of metadata, wherein the pathological profile comprises an information record comprising said plurality of metadata of said companion animal.

6. The method of claim 1, wherein said predictive model comprises at least a supervised classifier machine learning model.

7. The method of claim 1, further comprising the step of providing to a user interface said cutaneous lesion score relative to said companion animal.

8. The method of claim 1, further comprising the step of:
determining a respective size associated with each lesion of said companion animal,
normalizing the respective size associated with each lesion by a size of said companion animal, and
assessing a severity of the atopic dermatitis condition based on the cutaneous lesion score and the respective normalized size associated with each lesion.

9. The method of claim 1, further comprising the step of training the assessment module, using at least the plurality of previously acquired metadata relative to companion animals, selected in the data group (A), the method comprising:
extracting at least one feature from each previously acquired metadata,
associating at least said at least one feature to an animal cutaneous state, and
training the assessment module to learn said association.

10. The method of the preceding claim 2, wherein, said assessment module comprising at least one supervised classifier machine learning model, the method comprising the step of updating weights of the supervised classifier machine learning model according to said association between said at least one feature and said animal cutaneous state.

11. A device for determining a cutaneous lesion score of a companion animal suspected to have an atopic dermatitis condition, the device comprising an assessment module trained beforehand to learn features indicative of an atopic dermatitis condition in a companion animal, based at least on a plurality of previously acquired metadata relative to companion animals, selected in a data group (A) comprising at least animal data including two or more of at least (i) breed, (ii) species, (iii) gender, (iv) weight, (v) spayed or neutered status, (vi) age, (vii) age at onset, (viii) body condition, (ix) health status, (x) lifestyle, (xi) habitat, (xii) coat information, (xiii) activity level, (xiv) a biological value from a biological sample, (xv) age at onset and (xvi) dermatitis history, and derm area data corresponding to affected or unaffected derm area including two or more of at least (i) inguinal, (ii) axillar, (iii) ventral chest, (iv) perineal/genital, (v) ventral neck, (vi) pinnae, (vii) periorbitar, (viii) perioral, (ix) flexoral elbow, (x) fore feet, (xi) flexoral tarsal, (xii) hind feet and (xiii) nonspecific atopic area, said trained assessment module being configured to operate on the companion animal data, wherein the companion animal data includes a plurality of metadata relative to said companion animal, including two or more animal data and two or more derm area data listed in the data group (A), and to generate a cutaneous lesion score indicative of at least one cutaneous state of said companion animal.

12. The device of claim 11, comprising an acquisition module for acquiring said companion animal data.

13. Computer program product for determining a cutaneous lesion score of a companion animal suspected to have an atopic dermatitis condition, using an assessment module trained beforehand to learn features indicative of an atopic dermatitis condition in a companion animal, based at least on a plurality of previously acquired metadata relative to companion animals, selected in a data group (A) comprising at least animal data including two or more of at least (i) breed, (ii) species, (iii) gender, (iv) weight, (v) spayed or neutered status, (vi) age, (vii) age at onset, (viii) body condition, (ix) health status, (x) lifestyle, (xi) habitat, (xii) coat information, (xiii) activity level, (xiv) a biological value from a biological sample, (xv) age at onset and (xvi) dermatitis history, and derm area data corresponding to affected or unaffected derm area including two or more of at least (i) inguinal, (ii) axillar, (iii) ventral chest, (iv) perineal/genital, (v) ventral neck, (vi) pinnae, (vii) periorbitar, (viii) perioral, (ix) flexoral elbow, (x) fore feet, (xi) flexoral tarsal, (xii) hind feet and (xiii) nonspecific atopic area, the computer program product comprising a support and stored on this support instructions that can be read by a processor, these instructions being configured to:
a) receiving metadata relative to said companion animal, including two or more animal data and two or more derm area data listed in the data group (A),
b) encoding said metadata into a metadata vector,
c) operating said trained assessment module on said metadata vector, wherein the operating comprises inputting said metadata vector into a predictive model,
d) based on an analysis of said metadata vector by said predictive model associated with said assessment module, generating a cutaneous lesion score indicative of at least one cutaneous state of said companion animal, and
e) based on the cutaneous lesion score indicative of the at least one cutaneous state of said companion animal, assessing whether said companion animal has an atopic dermatitis condition.

14.-17. (canceled)
